(19) United States
(12) Patent Application Publication
(10) Pub. No.: US 2016/0113826 A1
(43) Pub. Date: Apr. 28, 2016

(54) ABSORBENT ARTICLE HAVING PERFORMANCE SIGNALS

(71) Applicant: The Procter & Gamble Company, Cincinnati, OH (US)

(72) Inventors: Xiaoxin LIU, Beijing (CN); Fancheng WANG, Beijing (CN); Limin SONG, Beijing (CN); Kazushige KISHIDA, Singapore (SG); Wolfgang Werner Hans DOMEIER, Beijing (CN); Li TANG, Beijing (CN)

(21) Appl. No.: 14/870,512

(22) Filed: Sep. 30, 2015

(30) Foreign Application Priority Data
Oct. 27, 2014 (WO) CN2014/089529

(51) Int. Cl. A61F 13/84

(52) U.S. Cl. CPC A61F 13/8405; A61F 2013/8497

(57) ABSTRACT

The present invention relates to an absorbent article comprising a plurality of first colored areas and a second colored area which are viewable by a user from a body-facing side of a topsheet, wherein the second colored area surrounds at least some of the first colored areas, a delta E between the first colored areas and the second colored area is at least about 4.5, an average mean area of the first colored areas is not smaller than about 0.20 mm², and a density of the first colored areas is about 4-24 areas/cm².

### FIELD OF THE INVENTION

The present invention relates to absorbent articles having signals to communicate performances and properties of the absorbent articles.

### BACKGROUND OF THE INVENTION

Absorbent articles such as sanitary napkins, pantiliners, tampons, absorbent interlabial devices, disposable diapers, incontinence products, and bandages are designed to absorb and retain liquid and other discharges from the human body and to prevent body and clothing soiling.

Through the use of innovative materials for components such as a topsheet, secondary topsheet, absorbent gelling materials and breathable backsheets composing absorbent articles, technologies in absorbent articles, and particularly sanitary napkins, have drastically advanced to provide women with products that absorb menses and other fluids away from a woman's body. However, much of these technologies are often hidden and therefore not viewable by a user. Even when viewable, absorbent components often do not readily or visually communicate to a user the existence of the enhanced technologies.

Important design criteria of topsheets include increase of absorbency and/or breathability of topsheets. When absorbency of the topsheets is not sufficient, users may not feel dry and discomfort may increase. When the topsheets are not breathable enough, it may cause skin troubles and users' discomfort may increase.

One approach for improving absorbency and/or breathability is forming apertures on a topsheet. Although apertured topsheets have generally reduced fluid pendency on topsheets and improved fluid penetration and absorbency and/or breathability, such improved topsheet properties have not been often sufficiently perceived by users.

It is believed that perceptions of absorbency and/or breathability of a material may be affected by visual signals, i.e., its visual appearance. It is believe that, if a material looks relatively absorbable or breathable to a person, it is much more likely that the person will perceived it as having relative absorbency and breathability as well. Visual impressions of absorbency or breathability may be affected by a variety of features and properties, including but not limited to color, and macroscopic physical surface features formed on a topsheet or any components of an absorbent article.

For example, one approach to enhancing perceived absorbency has involved forming a colored region on or below a top surface of an absorbent article that is visible through a topsheet to provide for a perception of depth and greater fluid storage capacity within the absorbent article. WO2003/53313 discloses an absorbent article having a multi-tone color signal of at least one color to create a perception of depth and absorbency by a user viewing the topsheet surface of the absorbent article.

Another approach to enhancing perceived absorbency or breathability has involved forming apertures having a specific mean area and density. WO2012/51467 discloses aperture parameters visible from the top surface of a sanitary napkin that creates an improved visual texture of the surface.

While approaches described above have had varying degrees of success, but have left room for improvement in effectively communicate and engender in a user the recognition of better protection and enhanced functioning of an absorbent article.

### SUMMARY OF THE INVENTION

The present invention relates to an absorbent article comprising a plurality of first colored areas and a second colored area which are viewable by a user from a body facing surface of a topsheet, wherein the second colored area surrounds at least part of the first colored areas, a delta E between the first colored areas and the second colored area is at least about 4.5, an average mean area of the first colored areas is not smaller than about 0.20 mm², and a density of the first colored areas is about 4-24 areas/cm².

These and other features, aspects, and advantages of the present invention will become evident to those skilled in the art from a reading of the present application.

### BRIEF DESCRIPTION OF THE DRAWINGS

The above-mentioned and other features and advantages of the present application, and the manner of attaining them, will become more apparent and the disclosure itself will be better understood by reference to the following description of non-limiting forms of the disclosure taken in conjunction with the accompanying drawings, wherein:

FIG. 1 is a perspective view of an absorbent article.

FIG. 2A is a top view of an absorbent article of the present invention.

FIG. 2B is a magnified image of a top surface of the absorbent article of FIG. 2A.

FIG. 3A is a top view of another absorbent article of the present invention.

FIG. 3B is a magnified image of a top surface of the absorbent article of FIG. 3A.

FIG. 4 is a top view of another absorbent article of the present invention.

FIG. 5A is a top view of a comparative absorbent article.

FIG. 5B is a magnified image of a top surface of the absorbent article of FIG. 5A.

FIG. 6A is a top view of a commercially available sanitary napkin.

FIG. 6B is a magnified image of a top surface of the absorbent article of FIG. 6A.

FIG. 7A is a top view of another commercially available sanitary napkin.

FIG. 7B is a magnified image of a top surface of the absorbent article of FIG. 7A.

### DETAILED DESCRIPTION

Various non-limiting forms of the present application will now be described to provide an overall understanding of the present invention. One or more examples of these non-limiting embodiments are illustrated in the accompanying drawings. Those of ordinary skilled in the art will understand that the present invention described herein and illustrated in the accompanying drawings are non-limiting example forms and that the scope of the various non-limiting forms of the present application are defined solely by the claims. The features illustrated or described in connection with one non-limiting form may be combined with the features of other non-limiting forms. Such modifications and variations are intended to be included within the scope of the present application.

The term “absorbent articles” as used herein, include disposable diapers, sanitary napkins, panty liners, incontinence pads, interlabial pads, breast-milk pads, sweat sheets, animal-use excreta handling articles, animal-use diapers, and the like.

The term “body facing surface” as used herein, refers to the side of the absorbent article facing the body of the user when in use. The term “garment facing surface” refers to the opposite surface of the article.

The terms “body fluid(s),” or “the fluid” as used herein, include, but are not limited to menses, vaginal discharges, blood, sweat, and combinations of these substances.

The term ‘color’ as used herein includes any no-white color, i.e., black, red, blue, violet, orange, yellow, green, and indigo as well as any declination thereof or mixture thereof, and white color. As used herein “white” is defined as having L*>90, −2<a*<2, and −2<b*<2.

The term “component” of an absorbent article, as used herein, refers to an individual constituent of an absorbent article, such as a topsheet, acquisition layer such as a secondary topsheet, absorbent core or layers of absorbent cores, backsheets, and barriers such as barrier layers and barrier cuffs.

The terms “joined”, “bonded”, or “attached” as used herein, encompasses configurations whereby a component is directly secured to another component by affixing the component directly to the other component, and configurations whereby a component is indirectly secured to another component by affixing the component to intermediate member(s) which in turn are affixed to the other component.

### Absorbent Article

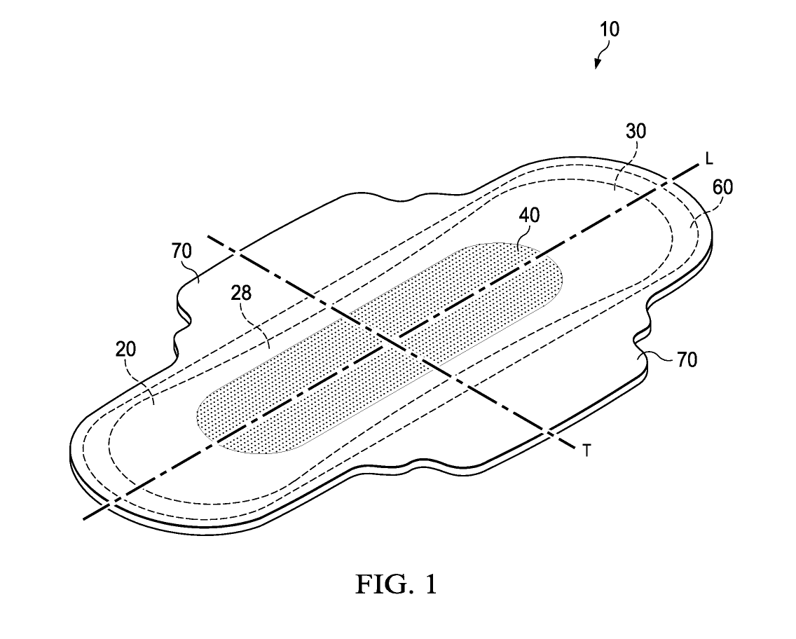

As shown in FIG. 1, an absorbent article 10 according to the present invention comprises a longitudinal centerline L, a transversal centerline T, and a topsheet 20 having a body facing surface 28 and a garment facing surface (not indicated in FIG. 1) positioned opposite to the body facing surface 28. The absorbent article 10 further comprises a backsheet (not indicated in FIG. 1) having a garment facing surface and a user facing surface positioned oppositely to the garment facing surface, the backsheet being joined to the topsheet 20. The absorbent article 10 also comprises an absorbent core 30 positioned between the topsheet 20 and the backsheet. The absorbent article 10 further comprises a plurality of first colored areas (not indicated in FIG. 1) and a second colored area (not indicated in FIG. 1) viewable from a side of the body facing surface 28 of the topsheet 20. The absorbent article 10 may comprise a secondary topsheet 60 and/or a pair of flaps 70. The absorbent article 10 may have a colored region 40. The colored region 40 is viewable from a side of the body facing surface 28 of the topsheet 20. In some embodiments, the absorbent article may comprise at least two colored regions.

The topsheet 20, the backsheet, and the absorbent core 30 can be assembled in a variety of well-known configurations.

The backsheet and the topsheet 20 can be secured together in a variety of ways. The topsheet 20 and the backsheet can be joined to each other by using an adhesive, heat bonding, pressure bonding, ultrasonic bonding, dynamic mechanical bonding, or a crimp seal. A fluid impermeable crimp seal can resist lateral migration (“wicking”) of fluid through the edges of the product, inhibiting side soiling of the user's undergarments.

As is typical for sanitary napkins and the like, the sanitary napkin can have panty-fastening adhesive disposed on the garment facing side of backsheet. The panty-fastening adhesive can be any of known adhesives used in the art for this purpose, and can be covered prior to use by a release paper, as is well known in the art. If flaps or wings are present, panty fastening adhesive can be applied to the garment facing side so as to contact and adhere to the underside of the user's panties.

Top Sheet

In the present application, a topsheet is the part of an absorbent article that is in contact with the user's skin. The topsheet may be joined to a backsheet, an absorbent core and/or any other layers as is known to those of skill in the art. Usually, the topsheet and the backsheet are joined directly to each other in some locations (e.g., on or close to the periphery of the absorbent article) and are indirectly joined together in other locations by directly joining them to one or more other components of the article.

The topsheet may be compliant, soft-feeling, and non-irritating to the user's skin. Further, a portion of, or all of, the topsheet may be liquid permeable, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. If the topsheet includes fibers, the fibers may be spunbond, carded, wet-laid, meltblown, hydroentangled, or otherwise processed as is known in the art.

The topsheet can be a polymeric film web. Polymeric film webs can be deformable. Deformable, as used herein, describes a material which, when stretched beyond its elastic limit, will substantially retain its newly formed conformation. Such deformable materials may be chemically homogeneous or heterogeneous, such as homopolymers and polymer blends, structurally homogeneous or heterogeneous, such as plain sheets or laminates, or any combination of such materials.

Deformable polymeric film webs that can be used can have a transformation temperature range in which changes in the solid state molecular structure of the material occur. Changes in the structure can include a change in crystalline structure and/or a change from solid to molten state. As a consequence, above the transformation temperature range, certain physical properties of the material are substantially altered. For a thermoplastic film, the transformation temperature range is the melt temperature range of the film, above which the film is in a molten state and loses substantially all previous thermo-mechanical history.

Polymeric film webs can comprise thermoplastic polymers having characteristic rheological properties which depend on their composition and temperature. Below their glass transition temperature, such thermoplastic polymers can be hard, stiff, and/or brittle. Below the glass transition temperature, the molecules are in rigid, fixed positions. Above the glass transition temperature but below the melt temperature range, thermoplastic polymers exhibit viscoelasticity. In this temperature range, the thermoplastic material generally has a certain degree of crystallinity, and is generally flexible and to some degree deformable under a force. The deformability of such a thermoplastic is dependent on the rate of deformation, amount (dimensional quantity) of deformation, length of time it is deformed, and its temperature. In one embodiment, processes can be utilized to form materials comprising thermoplastic polymers, especially thermoplastic film, which are within this viscoelastic temperature range.

Polymeric film webs can comprise a certain amount of ductility. Ductility, as used herein, is the amount of permanent, unrecoverable, plastic strain which occurs when a material is deformed, prior to failure (rupture, breakage, or separation) of the material. Materials that can be used as described herein can have a minimum ductility of at least about 10%, or at least about 50%, or at least about 100%, or at least about 200%.

Polymeric film webs can include materials normally extruded or cast as films such as polyolefins, nylons, polyesters, and the like. Such films can be thermoplastic materials such as polyethylene, low density polyethylene, linear low density polyethylene, polypropylenes and copolymers and blends containing substantial fractions of these materials. Such films can be treated with surface modifying agents to impart hydrophilic or hydrophobic properties, such as imparting a lotus effect. As noted below, polymeric film webs can be textured or otherwise altered from a strictly flat, planar configuration.

The topsheet can be a nonwoven web. As used herein, the term “nonwoven web” refers to a web having a structure of individual fibers or threads which are interlaid, but not in a repeating pattern as in a woven or knitted fabric, which do not typically have randomly oriented fibers. Nonwoven webs or fabrics have been formed from many processes, such as, for example, meltblowing, spunbonding, hydroentangling, airlaid, wetlaid, through-air-dried paper making processes, and bonded carded web processes, including carded thermal bonding. The nonwoven webs can comprise unbonded fibers, entangled fibers, tow fibers, or the like. Fibers can be extensible and/or elastic, and may be pre-stretched for processing. Fibers can be continuous, such as those produced by spunbonded methods, or cut to length, such as those typically utilized in a carded process. Fibers can be absorbent, and can include fibrous absorbent gelling materials. Fibers can be bicomponent, multiconstituent, shaped, crimped, or in any other formulation or configuration known in the art for nonwoven webs and fibers. The nonwoven webs comprising polymer fibers having sufficient elongation properties to be formed into an apertured web. In general, the polymeric fibers can be bondable, either by chemical bond (e.g. by latex or adhesive bonding), pressure bonding, or thermal bonding. If thermal bonding techniques are used in the bonding process described below, a certain percentage of thermoplastic material, such as thermoplastic powder or fibers can be used

The topsheet can be a composite or a laminate of two or more precursor webs, and can comprise two or more nonwoven webs or a combination of polymer films, nonwoven webs, woven fabrics, paper webs, tissue webs, or knitted fabrics.

The Topsheet can also optionally include colorants, such as pigment, lake, toner, dye, ink or other agent used to impart a color to a material. Suitable pigments herein include inorganic pigments, pearlescent pigments, interference pigments, and the like.

Any portion of the topsheet may be coated with a lotion and/or a skin care composition as is generally disclosed in the art.

The topsheet may comprise a plurality of apertures to ease penetration of fluids and/or air therethrough. The size of at least the primary apertures may be determined to achieve the desired fluid and/or air penetration performance and other performances expected by weares. If the apertures are too small, the fluids may not pass through the apertures, either due to poor alignment of the fluid source and the aperture location or due to runny fecal masses, for example, having a diameter greater than the apertures. If the apertures are too large, the area of skin that may be contaminated by “rewet” from the article is increased.

The topsheet may comprise a plurality of embossments to provide a more cloth like appearance.

The topsheet may have a printed area either on the body facing surface or a garment facing surface.

The topsheet may be formed of any basis weight. However, relatively higher basis weight, while having relatively greater apparent caliper and loft, also has relatively greater cost. On the other hand, in some embodiments, the basis weight of the multilayered nonwoven web may be high enough such that the topsheet can mask the color of a colored region positioned below the topsheet to enable first colored areas and a second colored area in the absorbent article to have a delta of at least 2.5 to be explained in detail below. Suitable basis weight for nonwoven or polymeric film for the topsheet of the present invention have been found to be 200 gsm or less, or from 7 gsm to 70 gsm, or from 10 gsm to 50 gsm, or from 12 gsm to 30 gsm.

In one embodiment, an absorbent article comprises a topsheet and a printed area, and the topsheet comprises a plurality of apertures and/or a plurality of embossments wherein at least part of the plurality of apertures and/or the plurality of embossments overlying the printed area, and the color of a first colored areas is perceived through the apertures and/or embossments. In such an embodiment, to obtain the desired degree of difference in color between the first colored areas and the second colored area in an absorbent article of the present invention, it is preferred that at least the part of the topsheet having a plurality of apertures and/or a plurality of embossments and overlying the colored region, when viewed from the body facing surface, has a sufficient basis weight such that the color contrast viewable from a body facing surface of the topsheet between the first colored areas defined by the apertures or embossments and the second colored area surrounding at least some of the first colored areas is increased by highly masking color of the second colored area.

Absorbent Core

An absorbent core of an absorbent article serves to store bodily fluids discharged during use. The absorbent core can be manufactured in a wide variety of sizes and shapes, and may be profiled to have different thickness, hydrophilic gradients, superabsorbent gradients, densities, or average basis weights at different positions across the face of the product.

An absorbent core may have a fluid distribution layer as well as a fluid storage layer. The fluid distribution layer transfers received fluid both downwardly and laterally, and generally has more permeability and less capillarity than the fluid storage layer.

In addition to conventional absorbent materials such as creped cellulose wadding, fluffed cellulose fibers, wood pulp fibers also known as airfelt, and textile fibers, the fluid storage layer often includes superabsorbent material that imbibe fluids and form hydrogels. These materials are typically capable of absorbing large quantities of body fluids and retaining them under moderate pressures. The fluid storage layer of the absorbent core can be made solely of superabsorbent material, or can include such materials dispersed in a suitable carrier such as cellulose fibers in the form of fluff or stiffened fibers.

Backsheet

The backsheet that covers the lower side of the absorbent core prevents the fluids in the absorbent core from wetting articles that contact the sanitary napkin, such as undergarments. Accordingly, the backsheet can be made from a liquid impervious thin film or a liquid impervious but vapor pervious film/nonwoven laminate, a microporous film, an apertured formed film, or other polymer film that is vapor permeable, or rendered to be vapor permeable, but substantially impervious to fluid.

First Colored Area and Second Colored Area

The absorbent article of the present invention comprises a plurality of first colored areas and a second colored area surrounding at least some of the first colored areas both of which are viewable by a user from a side of a body facing surface of a topsheet.

A delta E between the first colored areas and the second colored area is at least about 4.5 as determined by the Delta E Measurement disclosed herein below. In certain embodiments, the delta E between the first colored areas and the second colored area is at least about 4.5, or at least about 5.0, or at least about 6.0. It has been found that a delta E of 4.5 is already sufficient to provide an obvious color difference for wearers to sufficiently perceive breathability and/or absorbency. However, as delta E increases, the perception of breathability and/or absorbency may increase.

In addition, the first colored areas can have an average mean area in the range of from about 0.20 mm² to about 2 mm² The first colored areas can have an average mean area greater than about 0.20 mm², or greater than about 0.30 mm², or greater than about 0.40 mm² to communicate the depth perception.

Further, a density of the first colored areas is about 4-24 areas/cm², or about 6-24 areas/cm², or about 8-21 areas/cm², or about 10-13 areas/cm². If the density of the first colored areas is too small, the apertures in the absorbent article may not have connections to show a sufficient absorbency and/or breathability benefits. If the density is higher than 24, a pattern comprising the first areas may look very busy and the concern of leakage or other potential negative performances may be raised.

The first colored areas and the second colored area can be arranged uniformly to form a regular or irregular shape, or can be arranged non-uniformly to form various shapes.

Such arrangement of the first colored areas and the second colored area can engender in a user the recognition of better protection and enhanced functioning.

Generally, it may be desirable that the first colored areas have a more intense and/or darker color compared to the second colored area.

The first colored areas are of non-white color. The second colored area may be of non-white color or white.

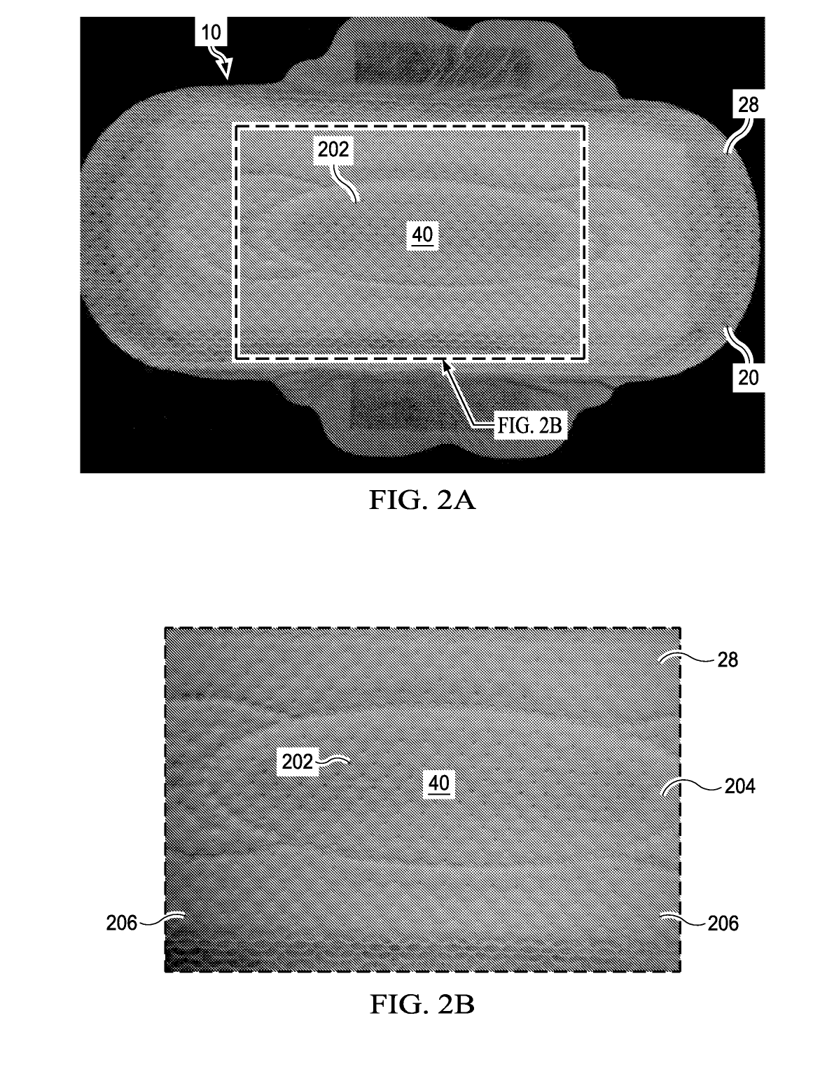

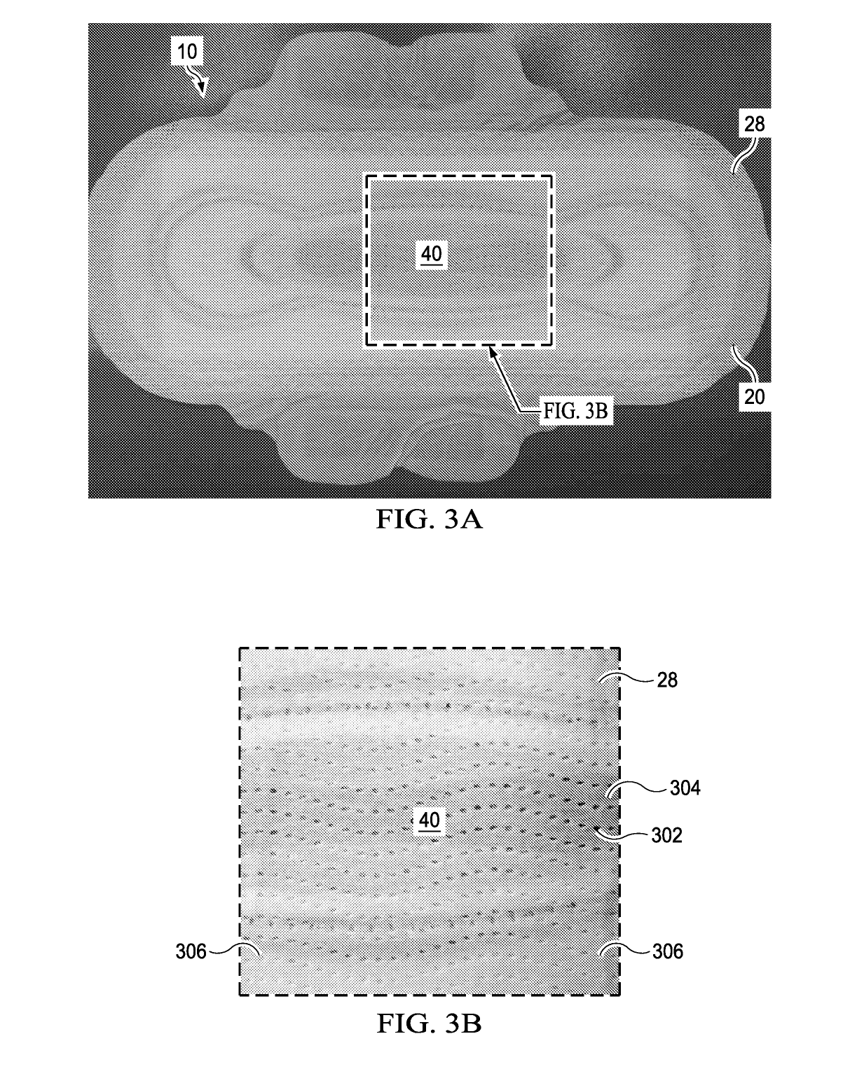

In one embodiment, as shown in FIGS. 2A-3B, an absorbent article 10 of the present invention comprises a plurality of first colored areas 202, 302 in a topsheet 20 of the absorbent article 10 wherein at least part of the apertures are formed overlying a colored region 40 positioned below the topsheet 20. The absorbent article 10 of the present invention further comprises a second colored area 204, 304 surrounding the first colored areas 202, 302. Referring to FIGS. 2A and 2B, apertures 206 overlying the colored region 40 define the first colored areas 202, and the color of the colored regions is visible through the apertures 206 from the body facing surface 28 of the topsheet 20, which is considered a color of the first colored areas 202. The area in the body facing surface 28 of the topsheet 20 surrounding the first colored areas 202, overlying the colored region 40 is the second colored area 204. Referring to FIGS. 3A and 3B, apertures 306 overlying the colored region 40 define the first colored areas 302, and the color of the colored regions is visible through the apertures 306 from the body facing surface 28 of the topsheet 20, which is considered a color of the first colored areas 302. The area in the topsheet 20 surrounding the first colored areas 302, overlying the colored region 40 is the second colored area 304.

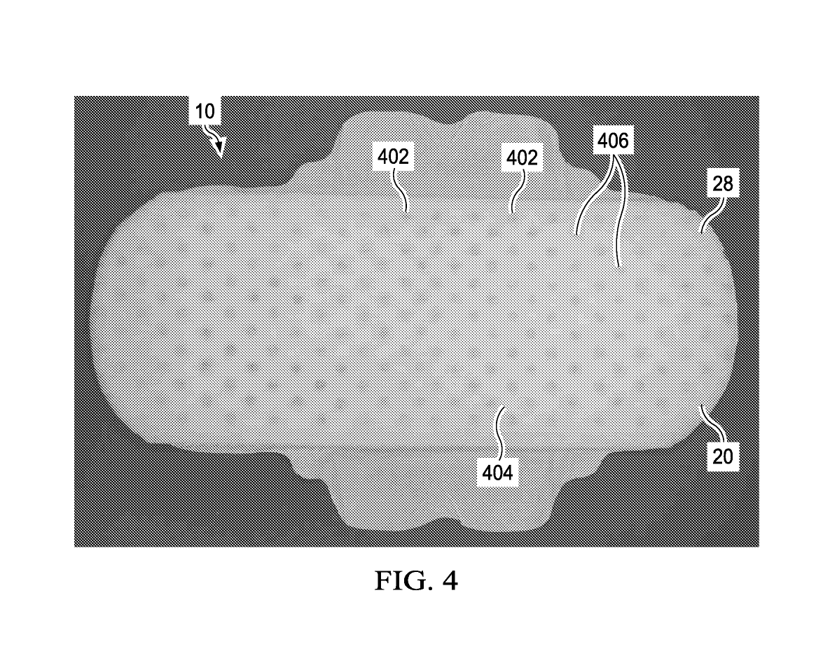

In another embodiment, an absorbent article of the present invention comprises a patterned print comprising a plurality of first printed areas in non-white color which defines a plurality of first colored areas, and a second colored area which surrounds at least some of the first colored areas. Referring to FIG. 4, an absorbent article of the present invention comprises a patterned print comprising a plurality of first printed regions 406 which defines a plurality of first colored areas 402, and a second colored area 404 which surrounds at least some of the first colored areas 402. The second colored area 404 can be white or non-white. The patterned print may further comprise a second printed area in non-white color which surrounds at least some of the first printed areas. The second printed area defines the second colored area. The patterned print may be printed on any surface of a component of the absorbent article of the present invention, for example on a surface of a topsheet, an absorbent core, and/or an optional insert or secondary topsheet. It is also possible that the plurality of first printed areas and the second printed area are printed in different components of the absorbent article, respectively, or different surfaces of the same or different components of the absorbent article, respectively.

In another embodiment, an absorbent article 10 of the present invention comprises a plurality of first colored areas defined by embossments formed in the topsheet 20 toward a bottom surface of the absorbent article 10 wherein at least part of the embossments are formed overlying a colored region 40 positioned below the topsheet 20 or a garment facing surface of the topsheet 20. Embossed areas overlying the colored region 40 define the first colored areas, and the color of the colored regions 40 visible through the embossed areas from the body facing surface 28 of the topsheet 20 is considered a color of the first colored areas. An area in a body facing surface of the topsheet 20 surrounding the first colored areas overlying the colored region 40 is a second colored area. Due to this embossment, the color of the colored region 40 below the topsheet 20 or on a garment facing surface of the topsheet 20 becomes more discernible from a body facing surface 28 of the topsheet 20 in the embossed areas. When viewing the body facing surface 28 of the topsheet 20, the color of the un-embossed area surrounding some of the embossed areas, the second colored area, the topsheet 20 positioned above the colored region 40 refract the light which masks the color of the colored region 40 to a certain extent.

In certain embodiments, the second colored area includes at least part of a longitudinal centerline of the absorbent article.

The absorbent article of the present application may have a total area of a plurality of first colored areas in the range of about 1% to about 25%, about 3% to about 18%, about 5% to about 12%, about 6% to about 10%, specifically reciting all 0.1% increments within the above-specified ranges and all ranges formed therein or thereby. The % first colored areas is measured according to the Average Mean Area of First Colored Areas Measurement described herein.

Colored Region

The absorbent article may comprise a colored region with non-white color. The colored region can be positioned on any suitable component of the absorbent article such as a topsheet, an optional additional layer, an absorbent article and a backsheet, as long as the color of the colored region is visible on and/or through in a plurality of first colored areas of the absorbent article from a body facing surface of a topsheet of the absorbent article.

In one embodiment, the colored region forms some of a topsheet. In yet another embodiment herein, the colored region forms some of the absorbent core. In certain embodiments, the colored region may be provided on any surface of a suitable component of the absorbent article including, for example, on a garment facing surface of a topsheet and/or a surface of an absorbent core. In certain embodiments, the colored region may be provided on an additional layer such as a secondary topsheet or an insert positioned beneath the topsheet, for example, between the topsheet and the absorbent core, or between the topsheet and a secondary topsheet when the layer is an insert.

The colored region can comprise a single colored region or a plurality of colored regions. A plurality of colored regions can be disposed as overlapping areas of color to define a color gradation or change in intensity by virtue of the overlapping nature of the regions. Colored or shaded regions can be any shape, including shapes, such as ocean waves, oval, a sandglass and the like. The colors of colored or shaded regions can be uniform, or they can be varying shades or hues of one color, or they can be different colors.

In certain embodiments, the colored region can be made by printing, such as, for example, by known processes, such as gravure printing, offset printing, inkjet printing, and combinations thereof. Alternatively, the colored region can be made by dyeing, pigmenting, or any other suitable coloration techniques. In certain embodiments, the colored region can be made by melt-adding a colorant during extrusion.

Therefore, the colored region can be ink or dye printed, coated, sprayed, or otherwise disposed on, secondary topsheets, surge layers, acquisition layers, absorbent cores, and the like.

Secondary Topsheet

In certain embodiments, the absorbent article can include a secondary topsheet that can be interposed between the absorbent core and the topsheet, and serves to rapidly draw discharged body fluids, in particular menstrual fluids, through the adjacent permeable (primary) topsheet. This allows the surface of the primary topsheet adjacent the user of the absorbent article to remain relatively clean and dry.

### Test Methods

Condition all samples at about 23° C.±2 C.° and about 50%±2% relative humidity for 2 hours prior to testing.

Average Mean Area of First Colored Areas Measurement

Average Mean Area of the first areas of an absorbent article are measured as follows.

Digital images of the article are captured and quantitatively analyzed to measure average mean area of first colored areas. The imaging system includes a light-tight imaging booth equipped with top-mounted, diffused lighting, simulating CIE D65 standard daylight, and a top-mounted digital RGB color camera with lens system, capable of providing images of the article at a spatial resolution of 40 micrometers per pixel, or fewer than 40 um per pixel. The camera body and light sources are oriented so that they are directly above and on a surface or plane parallel to the article being imaged, such that shadowing effects and variations in illumination intensity are both minimized in the captured image. An example of a suitable light booth system is the DigiEye Imaging System (VeriVide Ltd., Leicester, UK), with a Nikon D3 camera and 105 mm Nikkor Lens, yielding 4256×2832 pixel, 8 Bit, RGB images.

The imaging system is white balanced prior to use using a spectral standard Grey Card (21.6 cm×27.9 cm X-Rite Color Checker White Balance Card). A standard reference chart of various grey levels and colors is used to further calibrate the system for grey level intensity and contrast, and may also be use to calibrate color balance (X-Rite Gretag Macbeth Mini Color Checker Chart, 7.6 cm×12.7 cm). All image capture settings used in the system, including illumination intensity, camera exposure time, contrast gamma, etc., when used together in combination are set such that the 3 lightest grey level areas on the Color Checker Chart Standard are clearly distinguishable from each other in captured images of the chart. Image capture settings are set such that any color or intensity details observable in the article under the naked eye are equally or more observable in the captured images. Spatial calibration of the system to determine the number of micrometers per pixel in captured images is achieved by imaging a certified linear reference scale or rule (American National Institute of Standards and Technology (NIST)).

The intact, unused article to be measured is spread out flat inside the imaging system, with its body facing side/absorptive side, upwards toward the camera. The article is secured in place to maintain the article's surface flat and level relative to the camera lens.

After all calibrations are complete, the imaging system is used to capture images of the article in the largest colored or printed area in main central fluid absorbent acquisition zone of test articles. Typically this zone will lie near the center of the article, along the center line of the article's longest axis. An image of each article is captured such that 6 square cm of this area(s) is imaged on each article. If less than 6 square cm of colored or printed areas are present in the article's loading zone, then an image covering at least 1 square cm of these areas should be captured.

Three to five replicate articles of any specific type are imaged and analyzed.

Captured RGB images are converted to grayscale prior to further processing and analysis. One or more regions of interest (ROI) are selected within each image, so that as much of the image as possible is within ROI(s), and so that each ROI is as large as possible, while excluding areas of the image that were not within a colored or printed zone on the article.

Each Region(s) of Interest (ROI) is thresholded and binarized, thus designating each pixel into one of two classes (termed Background and Foreground). Thresholding should be achieved using Otsu's Gray Thresholding Method, which is a widely used method of automatic thresholding to binarize grayscale images. Otsu's method is a reiterative computation which independently determines the threshold value for each image that minimizes the weighted within-class variances for two classes. Many image analysis software packages can be used to perform Otsu's Thresholding, including Image J, Matlab, Image Pro Plus and others. The mathematic algorithm can be found at http://en.wikipedia.org/wiki/Otsu's_method or in Nobuyuki Otsu (1979). “A threshold selection method from gray-level histograms”. IEEE Trans. Sys., Man., Cyber. 9: 62-66.

The binary images are then analyzed to identify the first colored areas using Connected Component Labeling Analysis. Connected Component Labeling groups pixels into components/first colored areas/blobs based on pixel connectivity, i.e. all pixels in a connected component share similar pixel intensity values and are in some way connected with each other.

Some of the first colored areas found may be spatially located in contact with the edges of the image ROI, and therefore are likely partially cropped by the edge of the ROI. Such first colored areas are to be excluded from subsequent first colored areas measurements.

Measurements are then made on the number and area of first colored areas in each ROI, from each article. These measurements include:
The total number of first colored areas found, expressed on per area basis within the ROI(s) (e.g., 20 first colored areas per square cm).
The mean area for the first colored areas in each ROI.
The percentage of the total ROI area that is classified as being first colored areas (e.g, 20%).

Delta E Measurement

The color difference between the first colored areas and the second colored area measurement is based on the CIE L*a*b*color system (CIELAB). A flat bed scanner capable of scanning a minimum of 24 bit color at 1200 dpi and has manual control of color management (a suitable scanner is an Epson Perfection V750 Pro from Epson America Inc., Long Beach Calif.) is used to acquire images. The scanner is calibrated against a color reflection target compliant to ANSI method IT8.7/2-1993 using color management software (a suitable package is MonacoEZColor available from X-Rite Grand Rapids, Mich.) to construct a scanner profile. The resulting calibrated scanner profile is opened within an imaging program that supports sampling in CIE L*a*b* (a suitable program is Photoshop S4 available from Adobe Systems Inc., San Jose, Calif.) to measure bonded and unbonded areas.

Turn on the scanner for 30 minutes prior to calibration. Place the IT8 target face down onto the scanner glass and close the scanner lid. Open the MonacoEZColor software and select acquire image using the Twain software included with the scanner. Within the Twain software deselect the unsharp mask setting and any automatic color correction or color management options that may be included in the software. If the automatic color management cannot be disabled, the scanner is not appropriate for this application. Acquire a preview scan at 200 dpi and 24 bit color. Insure that the scanned image is straight and first outer surface facing side-up. Crop the image to the edge of the target, excluding all white space around the target, and acquire the final image. The MonacoEZColor software uses this image to compare with included reference files to create and export a calibrated color profile compatible with Photoshop. After the profile is created the scan resolution (dpi) can be changed, but all other settings must be kept constant while imaging samples.

Identify the first outer surface of the multilayered nonwoven web that contains the bonded areas of interest. Remove a piece of the multilayered nonwoven web. For convenience of handing, the sample size may be a 75 mm by 75 mm piece, however, as will be appreciated by the person skilled in the art, smaller samples sizes can be used. If the multilayered nonwoven web needs to be removed from a product, such as an absorbent article, it may be necessary to use a cryogenic freeze spray (e.g. CytoFreeze, Control Company, TX) to remove the specimen from the product. Precondition samples at about 23° C.±2 C.° and about 50%±2% relative humidity for 2 hours prior to testing.

Open the scanner lid and place the specimen onto the scanner glass with the first outer surface facing the glass. Cover the specimen with the white background (in this test method white is defined as having L*>94, −2<a*<2, and −2<b*<2) and close the lid. Acquire and import a scan of the specimen into Photoshop at 600 dpi and 24 bit color. Assign the calibrated scanner profile to the image and change the mode to Lab Color (“Lab Color” in Photoshop corresponds to the CIE L*a*b* standard). Select the “eyedropper” color selection tool. Set the sampling size of the tool to include as many pixels as possible within a bonded area without including pixels from adjacent unbonded areas. Using the eyedropper tool measure and record L*a*b* values in 10 different bonded areas in the nonwoven image. Average the 10 individual L*a*b* values and record as L₁, a₁, and b₁ respectively. Repeat the measure in like fashion for 10 different unbonded areas in the nonwoven image, and record the averaged values as L₂, a₂ and b₂. Calculate and report the color difference (delta E*) between the bonded and unbonded areas using the following equation:

delta E*=\sqrt{(L₂*−L₂*)²+(a₂*−a₁*)+(b₂*−b₁*)²}

and report to the nearest 0.01 units. A total of three substantially identical nonwoven webs are measured for each sample set. Average the three delta E** values and report to the nearest 0.1 unit.

### Example 1

### Sample Preparation

Samples 1-3

A nonwoven web comprising bicomponent fibers fibers having a unit weight of 40 g/m³ was used for a topsheet of Samples 1-3 sanitary pads. To prepare Sample 1 shown in FIGS. 2A and 2B, the nonwoven web was put into mechanical aperturing process to form apertures in the nonwoven web in a staggered pattern with an aperture density of 13 apertures/cm². A nonwoven web secondary topsheet was printed with blue-ink with the area of 20 cm² in the center of the secondary topsheet. Then, the topsheet, the secondary topsheet, an absorbent core and a backsheet were bonded to produce Sample 1. Sample 2 shown in FIGS. 3A and 3B was prepared according to the same process for Sample 1 except forming apertures in a wavy pattern with an aperture density of 8 apertures/cm². Sample 3 shown in FIG. 4 was prepared by printing first printed areas with blue-ink in a density of 4 printed areas/cm² throughout a body facing surface of a nonwoven secondary topsheet.

Comparative Sample 1

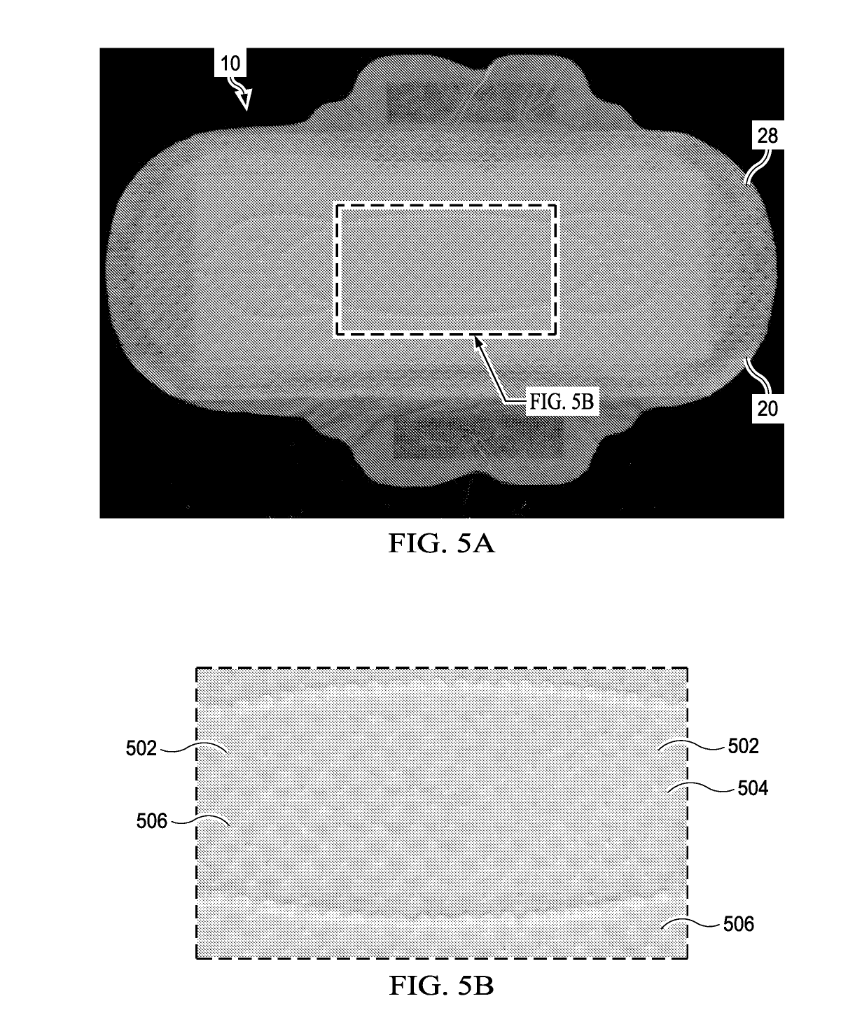

Comparative sample 1 shown in FIGS. 5A and 5B was prepared using the same materials as Sample 1 according to the process for Sample 1 except that the secondary topsheet was not printed. The apertures 506 define the first colored areas 502, and the area in the topsheet surrounding the first colored areas 502 is a second colored area 504.

### Example 2

### Measurement of Parameters of First Colored Areas and Second Colored Area

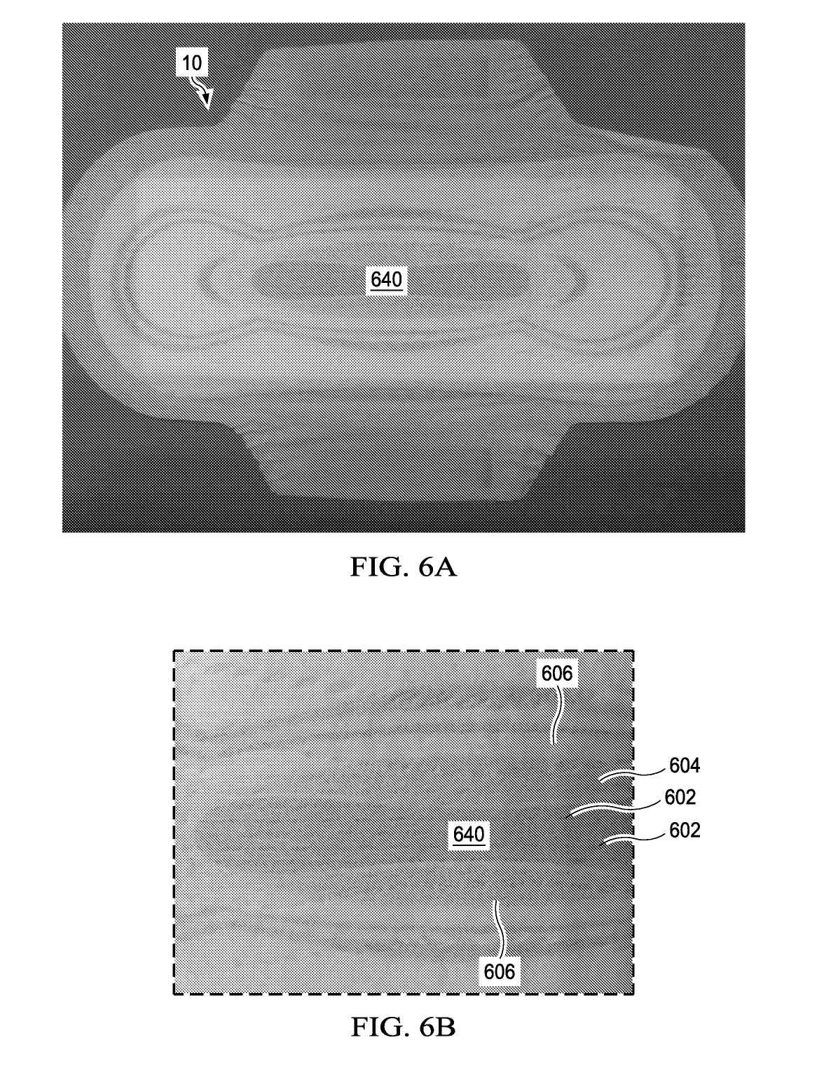

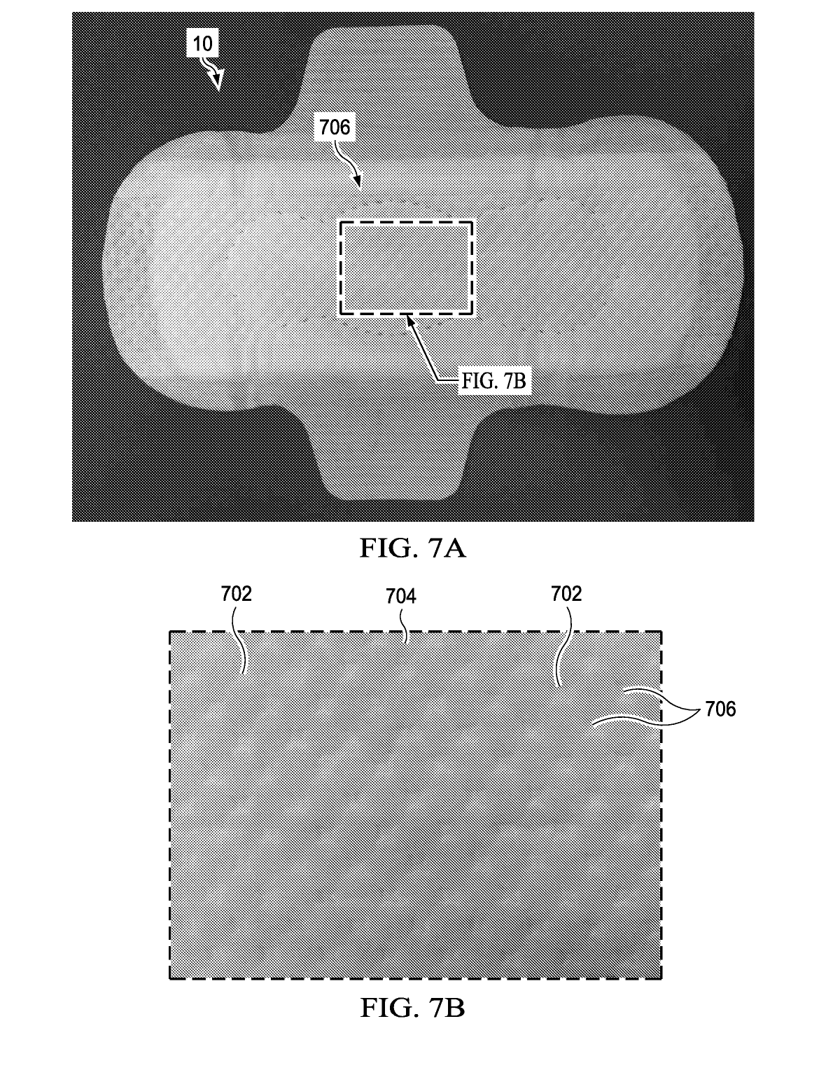

Delta E between the first colored areas and the second colored area, a density of the first colored areas and an average mean area of the first colored areas of Samples 1-3 and Comparative sample 1 were measured according to Test Method. Results are summarized in Table 1. Delta E of two areas, apetured or embossed areas and an area surrounding the apetured or embossed areas, in two marketed sanitary napkins were also measured. In Whisper product shown in FIGS. 6A and 6B, apertures 606 overlying the colored region 640 define the first colored areas 602, and the color of the colored region 640 is visible through the apertures from the body facing surface side of a topsheet, which is considered a color of the first colored areas 602. The area in the topsheet surrounding the first colored areas 602, overlying the colored region 640 is considered the second colored area 604. In Laurie F shown in FIGS. 7A and 7B, embossed areas 706 are considered defining the first colored areas 702, and the color visible through the apertures from the body facing surface side of a topsheet is considered a color of the first colored areas 702. The area in the topsheet surrounding the first colored areas 702 is considered the second colored area 704.

TABLE 1

| | | | | | | | Average |
|---|---|---|---|---|---|---|---|
| | | | | | | Number of | Mean Area |
| | | | | | | first | (mm²) of |
| | | | | | | colored | first colored |
| | | * | * | * | Delta E | area/cm² | areas |
| Sample 1 | First colored areas | 5.2 | 9.71 | 9.33 | 7.1 | 8 | 1.0 |
| | Second colored area | 7.99 | 4.58 | 5.27 | | | |
| Sample 2 | First colored areas | 4.6 | 8.88 | 9 | 6.3 | 13 | 0.7 |
| | Second colored area | 8.03 | 5.23 | 5.17 | | | |
| Sample 3 | First colored areas | 1.22 | 0.57 | 5.49 | 12.86 | 4 | 1.2 |
| | Second colored area | 2.28 | 0.90 | .07 | | | |
| Comparative | Apertured areas | 1.65 | 3.49 | 1.89 | 3.9 | 8 | 1.0 |
| Sample 1 | Area surrounding the | 1.16 | 0.32 | .38 | | | |
| | apertured areas | | | | | | |
| Whisper*¹ | First colored area | 4.99 | 10.35 | 8.34 | 2.13 | 9 | 1.3 |
| | Second colored area | 4.68 | 8.43 | 7.48 | | | |
| Laurier | Embossed areas | 4.04 | 0.88 | .28 | 1.18 | 6.5 | 1.3 |
| F*² | Area surrounding the | 4.77 | 0.59 | .40 | | | |
| | embossed areas | | | | | | |

*¹Whisper Pinckcess: Proctor & Gamble, China
*²Laurier F, Kao, Japan

### Example 3

### Breathability and Absorbency Measurement

30 Chinese consumers between ages 18-40 were asked to score products about perceptions of product breathability and absorbency. Sorting was conducted on a 1-10 scale: 10 means ideal performance, 1 means poorest performance. Results are shown in Table 2 below.

TABLE 2

| | | SD of | | SD of |
|---|---|---|---|---|
| | breathability | breathability | Absorbency | Absorbency |
| Sample 1 | 7.5 | 1.6 | 7.1 | 1.9 |
| Sample 2 | 7.6 | 1.6 | 7.1 | 1.9 |
| Comparative | 7.0 | 1.8 | 5.3 | 2.3 |
| sample 1 | | | | |
| Whisper*¹ | 6.1 | 2.1 | 5.5 | 2.0 |
| Laurier F*² | 6.5 | 1.7 | 6.6 | 2.2 |

*¹Whisper Pinckcess: Proctor & Gamble, China
*²Laurier F, Kao, Japan

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any embodiment disclosed or claimed herein or that it alone, or in any combination with any other reference or references, teaches, suggests or discloses any such embodiment. Further, to the extent that any meaning or definition of a term in this document conflicts with any meaning or definition of the same term in a document incorporated by reference, the meaning or definition assigned to that term in this document shall govern.

While particular embodiments of the present application have been illustrated and described, it would be obvious to those skilled in the art that various other changes and modifications may be made without departing from the spirit and scope of the present application. It is therefore intended to cover in the appended claims all such changes and modifications that are within the scope of this disclosure.

What is claimed is:

1. An absorbent article comprising;
a longitudinal centerline;
a liquid pervious topsheet having a body facing surface and a garment facing surface opposite the body facing surface,
a liquid impervious backsheet,
an absorbent core having a first surface and a second surface opposite to the first surface, and positioned between the topsheet and the backsheet,
a plurality of first colored areas, and
a second colored area surrounding at least some of the first colored areas,
wherein the plurality of first colored areas and the second colored area are viewable from the body facing surface of the topsheet,
wherein a delta E between the first colored areas and the second colored area is at least about 4.5, and
wherein the first colored areas have an average mean area of not smaller than about 0.20 mm², and a density of about 4-about 24 areas/cm².

2. The absorbent article of claim 1, wherein the absorbent article further comprises a layer having a first surface and a second surface opposite to the first surface below the topsheet.

3. The absorbent article of claim 2, wherein the layer is a secondary topsheet.

4. The absorbent article of claim 2, wherein the layer is an insert.

5. The absorbent article of claim 1, wherein the absorbent article further comprises a colored region.

6. The absorbent article of claim 5, wherein the absorbent article comprises the colored region in at least one surface selected from the garment facing surface of the topsheet, the first surface of the layer, the second surface of the layer, the first surface of the absorbent core, the second surface of the absorbent core, and any combination thereof.

7. The absorbent article of claim 5, wherein the colored region includes at least part of the longitudinal centerline.

8. The absorbent article of claim 1, wherein the delta E between the first colored areas and the second colored area is at least about 5.0.

9. The absorbent article of claim 1, wherein the plurality of first colored areas is different from the second colored area at least in one aspect selected from lightness, darkness, color, and any combination thereof.

10. The absorbent article of claim 1, wherein the plurality of first colored areas have an average mean area of not smaller than about 0.2 mm².

11. The absorbent article of claim 1, wherein the density of the first colored areas is about 8-about 24 areas/cm².

12. The absorbent article of claim 1, wherein the topsheet comprises a plurality of apertures defining the first colored areas.

13. The absorbent article of claim 1, wherein the topsheet comprises a plurality of embossed areas embossed toward the garment facing side of the topsheet, and the plurality of embossed areas defines the first colored areas.

14. The absorbent article of claim 1, wherein the absorbent article further comprises a plurality of first printed areas defining the first colored areas.

15. The absorbent article of claim 14, wherein the plurality of first printed areas is provided on one component of the absorbent article.

16. The absorbent article of claim 14, wherein the absorbent article further comprises a second printed area defining the second colored area.

17. The absorbent article of claim 16, wherein the first printed areas and the second printed area are provided in a different component of the absorbent article.

18. The absorbent article of claim 2, wherein the layer comprises a plurality of first printed areas defining the first colored areas.

19. The absorbent article of claim 1, wherein the topsheet is substantially white.

20. The absorbent article of claim 1, wherein the second colored area includes at least part of the longitudinal centerline.
